SEQUENCE LISTING (23 sequences)

SEQ ID NO: 1 - DNA, 20 bp, Human
atcccagatgtttctcaagt

SEQ ID NO: 2 - DNA, 21 bp, Human
atcccaaataaagtggtttct

SEQ ID NO: 3 - DNA, 20 bp, Human
tcagagcttaaactgggaag

SEQ ID NO: 4 - DNA, 21 bp, Human
agcaagaccctgtctcataga

SEQ ID NO: 5 - DNA, 21 bp, Human
gtcaacagaggcttgcatgta

SEQ ID NO: 6 - DNA, 28 bp, Human
ccccgcagtggatttggaaacagaaatg

SEQ ID NO: 7 - DNA, 28 bp, Human
ccccctcagtaagttaaaggattgcagg

SEQ ID NO: 8 - DNA, 23 bp, Human
tgtatttcatgtgtacattcgta

SEQ ID NO: 9 - DNA, 20 bp, Human
gattattttcactgtgggga

SEQ ID NO: 10 - DNA, 18 bp, Human
attccccaagtgaattgc

SEQ ID NO: 11 - DNA, 26 bp, Human
ggtagatagactggatagatagacga

SEQ ID NO: 12 - DNA, 20 bp, Human
aacaggatcaatggatgcat

SEQ ID NO: 13 - DNA, 20 bp, Human
cctctaataaatcccctctc

SEQ ID NO: 14 - DNA, 20 bp, Human
cctgttcctcccttatttcc

SEQ ID NO: 15 - DNA, 19 bp, Human
gaacacagactccatggtg

SEQ ID NO: 16 - DNA, 23 bp, Human
ggcatatttacaagctagtttct

SEQ ID NO: 17 - DNA, 20 bp, Human
atttgtctgtaattgccagc

SEQ ID NO: 18 - DNA, 18 bp, Human
gggaaccctcactgaatg

SEQ ID NO: 19 - DNA, 18 bp, Human
cagcgtttatttgcccaa

SEQ ID NO: 20 - DNA, 28 bp, Human
cccccctgacttggattgatctatctgt

SEQ ID NO: 21 - DNA, 29 bp, Human
ccccccgataaatacataggatggatgga

SEQ ID NO: 22 - DNA, 23 bp, Human
ggtgattttcctctttggtatcc

SEQ ID NO: 23 - DNA, 26 bp, Human
agccacagtttacaacatttgtatct

(19) United States
(12) Patent Application Publication
(10) Pub. No.: US 2008/0286773 A1
(43) Pub. Date: Nov. 20, 2008

(54) Method for Typing an Individual Using Short Tandem Repeat (Str) Loci of the Genomic Dna

(76) Inventor: Klaus Bender, Stadecken-Elsheim (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/816,895

(22) PCT Filed: Feb. 24, 2006
(86) PCT No.: PCT/EP2006/001701

§ 371 Date: May 26, 2008

(30) Foreign Application Priority Data
Feb. 24, 2005 (DE) 10 2005 008 583.0

(51) Int. Cl. C12Q 1/68

(52) U.S. Cl. 435/6

(57) ABSTRACT

The present invention relates to a novel STR typing strategy, which allows the simultaneous amplification and subsequent analysis of several (e.g. eleven) polymorphic systems with amplicon sizes of less than 270 bp. Thereby, after a PCR amplification the multiplex reaction is divided into two sets of STR multiplexes and analyzed separately. This multiplex system was particularly developed and tested for use in forensic investigations, where only limited amounts of DNA or only highly degraded DNA is available, for example, when the DNA is isolated from the roots of telogen hair.

The present invention relates to a novel STR typing strategy, which allows the simultaneous amplification and subsequent analysis of several (e.g. eleven) polymorphic systems with amplicon sizes of less than 270 bp. Thereby, after a PCR amplification the multiplex reaction is divided into two sets of STR multiplexes and analyzed separately. This multiplex system was particularly developed and tested for use in forensic investigations, where only limited amounts of DNA or only highly degraded DNA is available, for example, when the DNA is isolated from the roots of telogen hair. The present invention relates further to a respective kit for STR typing.

### BACKGROUND OF THE INVENTION

Today nearly solely features of the genomic DNA in the form of so called “short tandem repeat” (STR) polymorphisms are used for typing traces and persons [T. R. Moretti, A. L. Baumstark, D. A. Defenbaugh, K. M. Keys, J. B. Smerick, B. Budowle, Validation of short tandem repeats (STRs) for forensic usage: performance testing of fluorescent multiplex STR Systems and analysis of authentic and simulated forensic samples, J Forensic Sei 46 (2001) 647-660]. These are independently inherited autosomal short tandem repeat systems (STR systems), which after PCR amplification in multiplex PCR systems are separated, for example, by capillary electrophoresis.

The distance of the used primers to the recurring repeat untis results in constantly defined fragment lengths after PCR amplification. These lie between 100 and 400 bp in the commercially used multiplex systems. Thus, DNA pieces to by typed need to have at least the same length or need to be bigger [B. E. Krenke, A. Tereba, S. J. Anderson, E. Buel, S. Culhane, C. J. Finis, C. S. Tomsey, J. M. Zachetti, A. Masibay, D. R. Rabbach, E. A. Amiott, C. J. Sprecher, Validation of a 16-locus fluorescent multiplex System, J Forensic Sei 47 (2002) 773-785, E. A. Cotton, R. F. Allsop, J. L. Guest, R. R. Frazier, P. Koumi, I. P. Callow, A. Seager, R. L. Sparkes, Validation of the AMPFISTR SGM plus system for use in forensic casework, Forensic Sci Int 112 (2000) 151-161].

The investigation of minimal traces, like e.g. bone fragments, finger prints and telogen hairs, requires very effective typing methods, which combine a high sensitivity with the determination of as many as possible DNA features. The amplification is usually carried out under optimized conditions with the least DNA amounts (11 μl sample volume/34 PCR cycles). This method for the detection of “low copy number” DNA molecules has to be used for this kind of biological traces, because here only the least amounts of human DNA are to be expected. Also the amplification can only be carried out one time, because the extracted DNA is completely used up. It furthermore arises from the present methods that when three different fluorescence dyes are used as labels during the analysis at most five to six STR systems can be analyzed in one approach without the PCR fragments overlapping.

Nonetheless, in cases, where only a very limited amount of DNA and/or where the quality of the DNA is poor due to degradation, commercial multiplex kits result often in no DNA profiles or only in partial DNA profiles [P. M. Schneider, K. Bender, et al., STR analysis of artificially degraded DNA-results of a collaborative European exercise, Forensic Sci Int 139 (2004) 123-134].

A direct connection between amplification efficiency and amplicon size has clearly been shown [K. Bender, M. J. Farfan, P. M. Schneider, Preparation of degraded human DNA under controlled conditions, Forensic Sci Int 139 (2004) 135-140; D. T. Chung, J. Drabek, K. L. Opel, J. M. Butler, B. R. McCord, A study on the effects of degradation and template concentration on the amplification efficiency of the STR Miniplex primer sets, J Forensic Sci 49 (2004) 733-740]. In many cases hairs of the victim or of the potential criminal can be found at the crime site, from which, however, more then 90% are so called telogen hairs which have no attaching hair root cells. Amplicon sizes, which reach 100 to 400 bp, are commonly generated with the commercial STR kits. During typing of DNA from telogen hairs a loss of signal intensity is commonly observed in case of larger STR fragment sizes due to the fact that the DNA was fragmented in smaller pieces during hair development [H. Matsuda, K. Imaizumi, S. Kubota, S. Miyasaka, M. Yoshino, S. Seta, Technical Investigation of DNA extraction from single hair shaft, Rep Nat Res Inst Police Sci 50 (1997) 23-28]. In case that no hair root is present, only a typing of mitochondrial (mt) DNA (hair fragments) is possible, or the novel STR systems for shortened amplicon sizes can be used. For typing of difficult DNAs, i.e. highly degraded DNA, such as, for example, present in case of the extraction from hairs, shortened primers were, thus, developed in recent years. Thereby, the primers are advanced on the DNA sequence closer to the repeat unit, such that overall shorter DNA fragments can be analyzed. The minimal length of these fragments is at 60 to 250 bp [P. Grubwieser, R. Muhlmann, W. Parson, New sensitive amplification primers for the STR locus D2S1338 for degraded casework DNA, Int J Legal Med 117 (2003) 185-188; J. M. Butler, Y. Shen, B. R. McCord, The development of reduced size STR amplicons as tools for analysis of degraded DNA, J Forensic Sei 48 (2003) 1054-1064; P. Wiegand, M. Kleiber, Less is more-length reduction of STR amplicons using redesigned primers, Int J Legal Med 114 (2001) 285-287; Y. Shigeta, Y. Yamamoto, Y. Doi, S. Miyaishi, H. Ishizu, Evaluation of a method for typing the microsatellite D12S391 locus using a new primer pair and capillary electrophoresis, Acta Med Okayama 56 (2002) 229-236].

Until now either single PCR reactions or small multiplex PCRs were carried out [J. M. Butler, Y. Shen, B. R. McCord, The development of reduced size STR amplicons as tools for analysis of degraded DNA, J Forensic Sei 48 (2003) 1054-1064; P. Wiegand, M. Kleiber, Less is more-length reduction of STR amplicons using redesigned primers, Int J Legal Med 114 (2001) 285-287].

A further approach is the consecutive amplification of single STR systems, wherein the DNA is bound to a membrane (solid phase PCR) [A. Hellmann, U. Rohleder, H. Schmitter, M. Wittig, STR typing of human telogen hairs—a new approach, Int J Legal Med 114 (2001) 269-273]. This approach, which was particularly developed for typing of roots of telogen hair, uses a series of single STR typing steps, where the DNA extracted from the hair is fixed onto a membrane during the consecutive PCR reactions. This so called solid phase PCR, which was initially published for three STR systems, has now been expanded for up to seven STR systems plus amelogenin (H. Schmitter and A. Hellmann, personal communication). Although the test functions well, this procedure is time consuming and the success of typing highly depends on the quality of the membrane used for binding the DNA. To avoid these repetitive PCR amplification steps, short PCR multiplexes were developed by several work groups [J. M. Butler, Y. Shen, B. R. McCord, The development of reduced size STR amplicons as tools for analysis of degraded DNA, J Forensic Sci 48 (2003) 1054-1064; P. Wiegand, M. Kleiber, Less is more-length reduction of STR amplicons using redesigned primers, Int J Legal Med 114 (2001) 285-287; C. Meissner, personal communication]. As in all commercial STR multiplex kits the different STR systems are differentiated by their amplicon sizes and the fluorescence dye for the respective locus. In case that the maximum size of the amplified PCR products is limited to about 250 bp, only a small number of STR can be arranged in this size range for the typing of highly fragmented DNA.

Thus, it is a disadvantage that with the single PCR, only one STR system can be analyzed in case that only the least amounts of DNA are present. With the small multiplexes also only few STR systems can be analyzed. In many cases, to few systems are present that can be utilized in the data bases. In case of the solid phase PCR exists an increased contamination risk due to the frequent wash steps, furthermore carrying out the analysis strongly depends on the quality of the membrane used.

Thus, the investigation of minimal traces, like e.g. bone fragments, finger prints and telogen hairs, requires very effective typing methods, which combine a high sensitivity with the determination of as many as possible DNA features.

In a first aspect thereof this problem is solved by the present invention by a method for typing an individual, that comprises the steps of a) providing genomic DNA of the individual; b) amplification of at least two short tandem repeat (STR) loci of the genomic DNA using, if applicable labelled, pairs of amplification primers, wherein at least one primer is provided with a binding group; c) separating the amplified STR fragments by means of the binding group in at least two fractions of amplificates; and d) separate detection of the STR fragments of the fractions.

By combining two (multiplex) PCR reactions, which are separated from each other in the course of the analysis, it is possible to achieve an improved quality of the STR analysis, in particular in case of least amounts of DNA. Furthermore, it is possible for the first time to analyze at least ten autosomal STR systems plus the gender-specific amelogenin system in one approach.

According to the present invention this is achieved in that in a preferred embodiment two smaller multiplex PCR reactions (one 5-plex and one 6-plex) are amplified together as a multiplex PCR. By using, for example, biotinylated PCR primers in one of the two small multiplexes its products can be separated after the PCR after binding to, for example, streptavidin-coated sepharose beads and separately assayed by capillary gel electrophoresis. Thus, the PCR products would indeed overlap in the multiplex, but not during the separation of the multiplexes and the separate capillary gel electrophoresis. The difference to the methods described above is, thus, the use of PCR primers provided with a binding group (e.g. biotinylated primers) in a subset of the multiplex reaction. This allows the amplification of PCR fragments that overlap in size, which could not be assayed together in the capillary gel electrophoresis without separation.

Advantageously, the invention allows the determination of as many as possible DNA features even from the least DNA traces in one approach. Thus, as little as possible trace material is used in order to allow a second independent analysis or to even allow the determination of several features in case of least amounts of DNA, where the whole isolated DNA has to be used. When compared to the successive amplification using the solid phase PCR according to e.g Hellmann et al. the present invention further saves a considerable amount of time and results in a considerably lowered contamination risk. When compared to other multiplex reactions the present invention allows a considerable increase in the number of DNA features that can be investigated.

A method of typing an individual according to the present invention is preferred, wherein the individual is a mammal, such as e.g. a human.

A method of typing an individual according to the present invention is further preferred, wherein the STR loci are selected from the group comprising D3S1358, D8S1179, D21S11, TH01, FGA, VWA, D2S1338, D12S391, TPOX, D5S818, D18S51, FES and amelogenin.

A method of typing an individual according to the present invention is even further preferred, wherein more than three, four, five, six, seven, eight, nine, ten or eleven STR loci are amplified. According to the present invention the amplification can be carried out using PCR and/or multiplex amplification.

A method of typing an individual according to the present invention is also further preferred, wherein the primers are labelled with a fluorescence dye. The fluorescence dye can by any suitable dye, in particular it is selected from the group comprising 6-FAM, JOE, NED and PET (red).

A method of typing an individual according to the present invention is also further preferred, wherein the binding group is selected from the group comprising biotin, streptavidin, a His tag, heat stable antigen and oligonucleotide. According to the present invention all binding groups can be used, that do not interfere with the amplification and are suitable for a subsequent separation of the fractions. This separation of the amplified STR fragments by means of the binding group can comprise a solid phase, which has, for example, biotin, streptavidin, antibodies or complementary oligonucleotides immobilized thereto. According to the present invention the solid phase can comprise a membrane, sepharose beads or magnetic sepharose beads. Suitable other phase are well known to the person of skill in the art.

A method of typing an individual according to the present invention is then preferred, wherein the genomic DNA is derived from blood, blood constituents, sperm and/or telogen hairs.

A method of typing an individual according to the present invention is further preferred, wherein the detection of the STR fragments of the fractions comprises a length determination of the fragments, for example by capillary gel electrophoresis. Suitable other detection methods are well known to the person of skill in the art.

A method of typing an individual according to the present invention is even further preferred, wherein as primer pairs at least two pairs are used that are selected from the group of SEQ ID NOs. 1-3; 4 and 5; 6 and 7; 8 and 9; 10 and 11; 12 and 13; 14 and 15; 16 and 17; 18 and 19; 20 and 21; and 22 and 23 (see Table 1).

A further aspect of the present invention relates then to a method of typing an individual, which further comprises an identification of the individual on the basis of the detected STR fragments.

A further aspect of the present invention relates then to a diagnostic kit, comprising at least two pairs of STR primers for performing a method as described above, optionally with further materials and excipients. Respective materials and excipients are, for example, PCR reagents, buffers, solid phases, instruction manuals, dyes and others.

A last aspect of the present invention relates then to the use of the method as described above or of the kit as described above in the context of forensics.

A novel STR typing strategy was developed, which allows the simultaneous amplification and subsequent analysis of (e.g.) eleven polymorphic systems with amplicon sizes of less than 270 bp. The multiplex amplification reaction includes six STR loci from the European standard set of loci (ESS) for DNA data bases (D3S1358, D8S1179, D21S11, TH01, FGA and VWA) as well as four additional STR systems, which were selected due to their robustness (D2S1338, D12S391, TPOX and D5S818), together with the gender-specific locus amelogenin. After the PCR amplification the multiplex reaction is divided in two sets of STR multiplexes by using Biotin-labelled primers for only one of the sets. Using streptavidin-coated sepharose beads, for example, five STR systems (or also between two to nine) are separated from the remaining (e.g. six) systems, prior to analyzing them in two different runs on a capillary gel electrophoresis instrument. This multiplex system was particularly developed and tested for the use in forensic investigations, where only limited amounts of DNA or only highly degraded DNA is available, for example, when DNA is isolated from the roots of telogen hair.

In the present invention the inventors describe a method, wherein e.g. two 5-plex and 6-plex PCR reactions are combined in a big multiplex reaction. Ten STR systems plus amelogenin are co-amplified, with maximum fragment sizes of up to 262 base pairs (Table 2), and then divided in the two initial small multiplex reactions. The primer sequences were selected from published data [P. Grubwieser, R. Muhlmann, W. Parson, New sensitive amplification primers for the STR locus D2S1338 for degraded casework DNA, Int J Legal Med 117 (2003) 185-188; Y. Shigeta, Y. Yamamoto, Y. Doi, S. Miyaishi, H. Ishizu, Evaluation of a method for typing the microsatellite D12S391 locus using a new primer pair and capillary electrophoresis, Acta Med Okayama 56 (2002) 229-236; J. M. Butler, Y. Shen, B. R. McCord, The development of reduced size STR amplicons as tools for analysis of degraded DNA, J Forensic Sci 48 (2003) 1054-1064; A. Hellmann, U. Rohleder, H. Schmitter, M. Wittig, STR typing of human telogen hairs—a new approach, Int J Legal Med 114 (2001) 269-273], obtained from a commercially available multiplex kit as well as provided by courtesy of Dr. Schmitter and Dr. Hellmann (Federal Criminal Police Office, Wiesbaden, Germany). In this novel multiplex approach, for example, five of 11 STR systems were amplified by using Biotin-labelled reverse primers. Thus, it is possible to separate the biotinylated amplicons from the multiplex PCR, for example, by using streptavidin-coated sepharose beads. Each of the two separated multiplexes was then analyzed on ABI Prism 310 and/or 3100avant Genetic Analyzers.

The present innovative approach combines at the same time the multiplex amplification of e.g. eleven loci with very short amplicons with the biochemical separation of the amplicons in e.g. two fractions of fragments for the subsequent electrophoretical analysis. Even though the e.g. biotin separation procedure requires somewhat more work and time, it has the major advantage of saving precious sample material in that it allows the simultaneous analysis of ten short amplicon STR systems from one single DNA aliquot. This method can be used in cases, where most of the commonly used multiplex STR kits are not able to produce reliable results. The multiplex includes six of the seven European STR loci for national DNA data banks and eight STR loci from the U.S. CODIS data bank [P. D. Martin, H. Schmitter, P. M. Schneider, A brief history of the formation of DNA databases in forensic science within Europe, Forensic Sci Int 119 (2001) 225-231]. The so called “BioPlex-11” multiplex PCR system which is described herein, with its high distinction (PD) value of 4.21×10¹² for all ten STRs and 5.24×10⁷ for the six European data bank STR systems, provides a significant and sensitive system for a powerful analysis of degraded and “low copy” DNA samples.

The present invention shall now be further clarified on the bases of the following examples by reference to the accompanying figures.

The Figures show:

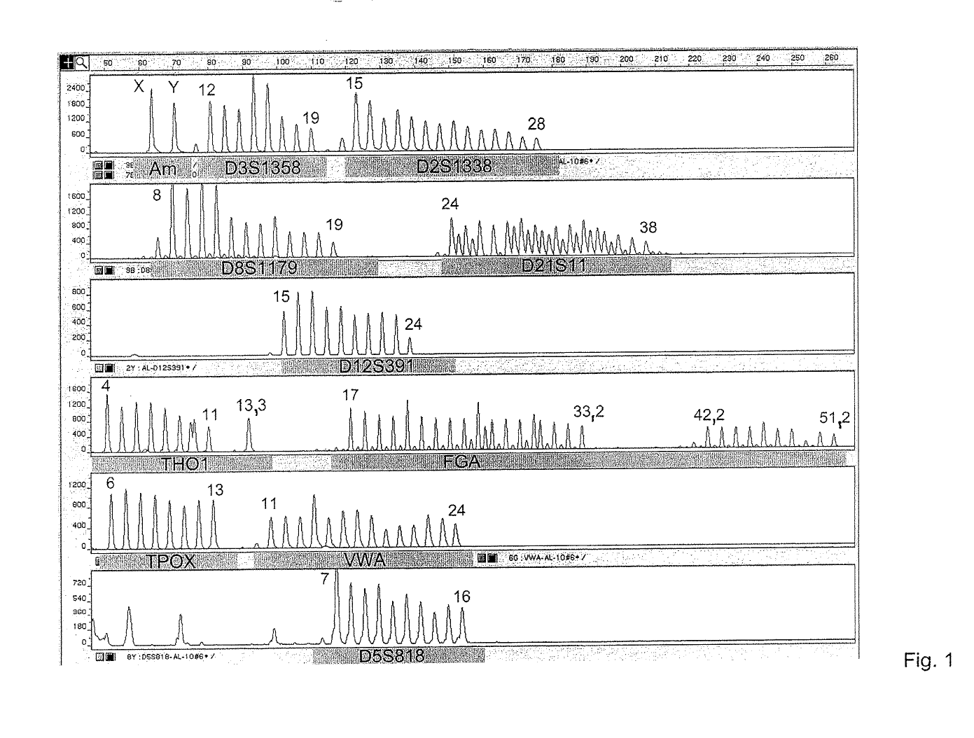

FIG. 1. The allele ladders for the novel Bioplex-11 were re-amplified from the allele standards of the SGM Plus™ (Applied Biosystems) or PowerPlex® 16 (Promega) kits and from the “self-assembled” D12S391 ladder. The three upper panels show the two 6-FAM-labelled STR systems D3S1358 and D2S 1338 together with amelogenin, the two JOE-labelled STR systems D8S1179 and D21S11, as well as the D12S391 system, which belongs to the 6-plex part of the multiplex. The three lower panels show the two 6-FAM-labelled STR systems TH01 and FGA, the two JOE-labelled STR systems TPOX and VWA, as well as the D5S818 system from the biotinylated 5-plex submultiplex.

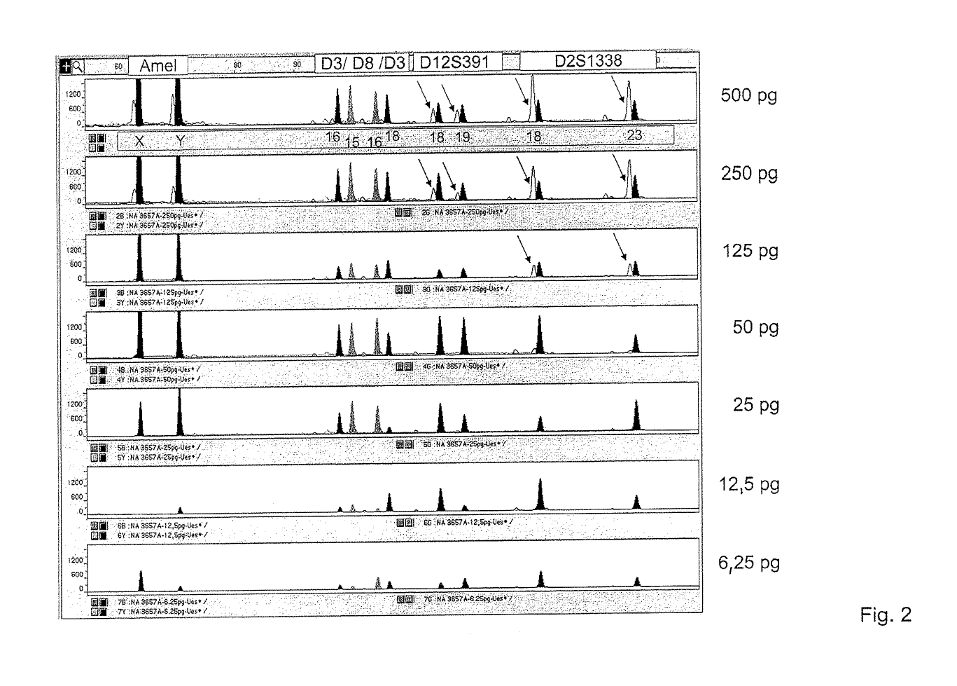

FIG. 2. Sensitivity analysis using successive dilutions of genomic DNA of 500 pg down to 6.25 pg. For a better view, the screenshot shows only the electrophorogram of the 5-plex part of the multiplex. PCR products were separated and detected on the ABI PRISM 310 Genetic Analyzer. Arrows show peaks, which show the N and N+1 fragments, which are typical for the over amplification of DNA samples.

TABLE 1

PCR primer sequences for amelogenin and STR systems. C-stretch sequences
for the elongation of the PCR product are underlined. The reference
sequences for the STR marker were obtained from GenBank ®
(http://cst1.nist.gov).

| | | | Primer sequences (5′-3′) |
|---|---|---|---|
| STR Locus | Primer | GenBank Accession Number | - SEQ ID NO. |
| Am | AX-F | K. M. Sullivan, A. et al., | ATC CCA GAT GTT TCT CAA |
| | AY-F | A rapid and quantitative | GT (SEQ ID NO. 1) |
| | AXY-R (6- | DNA sex test: | ATC CCA AAT AAA GTG GTT |
| | FAM) | fluorescence-based PCR | TCT (SEQ ID NO. 2) |
| | | analysis of X-Y | TCA GAG CTT AAA CTG GGA |
| | | homologous gene | AG |
| | | amelogenin, Biotechniques | (SEQ ID NO. 3) |
| | | 15 (1993) 636-638, 640- | |
| | | 631. | |
| D3S1358 | D3S1358vs- | NT_005667 | AGC AAG ACC CTG TCT CAT |
| | F (6-FAM) | | AGA |
| | D3S1358vs- | | (SEQ ID NO. 4) |
| | R | | GTC AAC AGA GGC TTG CAT |
| | | | GTA |
| | | | (SEQ ID NO.5) |
| D2S1338 | D2S1338vs- | AC010136 | CCC CGC AGT GGA TTT GGA |
| | F (6-FAM) | | AAC AGA AAT G |
| | D2S1338vs- | | (SEQ ID NO. 6) |
| | R | | CCC CCT CAG TAA GTT AAA |
| | | | GGA TTG CAG G |
| | | | (SEQ ID NO. 7) |
| D8S1179 | D8S1179vs- | AF216671 | TGT ATT TCA TGT GTA CAT |
| | F (JOE) | | TCG TA |
| | D8S1179vs- | | (SEQ ID NO. 8) |
| | R | | GAT TAT TTT CAC TGT GGG |
| | | | GA |
| | | | (SEQ ID NO. 9) |
| D21S11 | D21S11vs-F | M84567 | ATT CCC CAA GTG AAT TGC |
| | (JOE) | | (SEQ ID NO. 10) |
| | D21S11vs-R | | GGT AGA TAG ACT GGA TAG |
| | | | ATA GAC GA |
| | | | (SEQ ID NO. 11) |
| D12S391 | D12S391vs- | G08921 | AAC AGG ATC AAT GGA TGC |
| | F (NED) | | AT |
| | D12S391vs- | | (SEQ ID NO. 12) |
| | R | | CCT CTA ATA AAT CCC CTC |
| | | | TC |
| | | | (SEQ ID NO. 13) |
| THO1 | THO1vs-F | D00269 | CCT GTT CCT CCC TTA TTT CC |
| | (6-FAM) | | (SEQ ID NO. 14) |
| | THO1vs-R | | GAA CAC AGA CTC CAT GGT |
| | (Bio) | | G |
| | | | (SEQ ID NO. 15) |
| FGA | FGAvs-F (6- | M64982 | GGC ATA TTT ACA AGC TAG |
| | FAM) | | TTT CT |
| | FGAvs-R | | (SEQ ID NO. 16) |
| | (Bio) | | ATT TGT CTG TAA TTG CCA |
| | | | GC |
| | | | (SEQ ID NO. 17) |
| TPOX | TPOXvs-F | M68651 | GGG AAC CCT CAC TGA ATG |
| | (JOE) | | (SEQ ID NO. 18) |
| | TPOXvs-R | | CAG CGT TTA TTT GCC CAA |
| | (Bio) | | (SEQ ID NO. 19) |
| VWA | VWAvs-F | M25858 | CCC CCC TGA CTT GGA TTG |
| | (JOE) | | ATC TAT CTG T |
| | VWAvs-R | | (SEQ ID NO. 20) |
| | (Bio) | | CCC CCC GAT AAA TAC ATA |
| | | | GGA TGG ATG GA |
| | | | (SEQ ID NO. 21) |
| D5S818 | D5S818vs-F | AC008512 | GGT GAT TTT CCT CTT TGG |
| | (NED) | | TAT CC |
| | D5S818vs-R | | (SEQ ID NO. 22) |
| | (Bio) | | AGC CAC AGT TTA CAA CAT |
| | | | TTG TAT CT |
| | | | (SEQ ID NO. 23) |

### EXAMPLES

DNA extraction. Human genomic DNA was extracted from blood and forensic samples of telogen hair. Blood samples were extracted with the E.Z.N.A. Blood DNA Kit II (peqlab Biotechnologie GmbH, Germany). Control DNA of NA3657A cells was a courtesy of Dr. R. Szibor [R. Szibor, J. Edelmann, S. Hering, I. Plate, H. Wittig, L. Roewer, P. Wiegand, F. Cali, V. Romano, M. Michael, Cell line DNA typing in forensic genetics—the necessity of reliable standards, Forensic Sci Int 138 (2003) 37-43.], Institute of Forensic Medicine, University of Magdeburg, Germany. The NA9947A DNA sample was obtained from the PowerPlex® 16 Kit (Promega, Madison, USA). Hair samples of former cases were used. DNA from telogen hairs was extracted as previously described by Hellmann et al. [A. Hellmann, U. Rohleder, H. Schmitter, M. Wittig, STR typing of human telogen hairs—a new approach, Int J Legal Med 114 (2001) 269-273.]. Briefly, about one centimetre of a hair fragment, containing the telogen root, was digested in 500 μl TNca buffer. After purification by standard phenol/chloroform procedures the DNA was concentrated with a Microcon-30 microconcentrator (Millipore, Eschborn, Germany). The STR analysis of artificially degraded DNA was carried out by using aliquots of DNA from the P118 and HepG2 cell lines, which were stored from our collaborative European exercise of degraded DNA [P. M. Schneider, K. Bender, et al. STR analysis of artificially degraded DNA-results of a collaborative European exercise, Forensic Sci Int 139 (2004) 123-134, K. Bender, M. J. Farfan, P. M. Schneider, Preparation of degraded human DNA under controlled conditions, Forensic Sci Int 139 (2004) 135-140.].

PCR amplification. For the amplification of very short tandem repeat systems the commercially available Quiagen® Multiplex PCR Kit (Quiagen, Hilden, Germany) was used according to the instructions of the manufacturer with Applied Biosystems Gene®Amp PCR System 2400/2700 thermocyclers. The PCR was carried out in 25 μl reaction volumes, containing 11 μl template DNA. 12.5 μl multiplex PCR buffer [containing HotStar Taq® DNA polymerase, dNTP mix, 6 mM MgCl₂, 10 μM of each primer (Table 2) under the following conditions: initial denaturing at 95° C. for 15 min, 34 cycles of denaturing at 94° C. for 30 sec, primer annealing at 57° C. for 90 sec, elongation at 60° C. for 90 sec and a final elongation step at 60° C. for 30 min.

Amplification of allele ladders. Allele ladders from the commercially available SGM Plus™ (Applied Biosystems) or PowerPlex® 16 (Promega) kits were used as template. Ladder aliquots from the kits were diluted 1 to 1.000.000 and amplified in individual PCR reactions, using the same conditions as described for the multiplex PCR, except of a decreased number of 30 cycles. To show the accuracy of these ladders, the allele determinations of all loci were compared between the samples which were typed with the SGM Plus™ and the PowerPlex® 16 kit and the novel Bioplex-11 multiplex kit. All results were found to be identical (FIG. 1). The allele ladder for D21S391 was amplified from a ladder, which was used in a previous study [W. Waiyawuth, L. Zhang, C. Rittner, P. M. Schneider, Genetic analysis of the short tandem repeat system D12S391 in the German and three Asian populations, Forensic Sci Int 94 (1998) 25-31].

When the same fluorescence dye was used for more than one STR locus, all the allele ladders were so designed that they do not overlap. For example, the JOE-labelled systems D8S1179 and D21S11 (spanning from 76 to 120 bases and from 153 to 209 bases, respectively) and die 6-FAM-labelled systems TH01 and FGA (spanning from 56-95 bases and from 124-262 bases, respectively) are separated by about 30 bases. Some STR loci are only separated by a few base pairs, e.g. the 6-FAM-labelled systems D3S1358 and D2S1338 as well as the JOE-labelled systems TPOX and VWA. To avoid false allele signals for alleles outside the ladder area, the fragment sizes of the adjacent STR systems lie one or two bases outside of the tetramer reading frame for the allele determination. This was achieved by increasing the sizes of the amplicons of the larger systems by adding of up to six bases onto the 5′ end of both primers (see Table 2).

TABLE 2
Allele areas and the respective fragment sizes
for amelogenin and all STR systems;

| | | PCR Fragment | |
|---|---|---|---|
| STR Locus | Allele Areas | (bp) | Fluorescence Dye |
| Amelogenin | X, Y | 69, 75 | 6-FAM (blue) |
| D3S1358 | 12-19 | 86-114 | 6-FAM (blue) |
| D2S1338 | 15-28 | 127-179 | 6-FAM (blue) |
| D8S1179 | 8-19 | 76-120 | JOE (green) |
| D21S11 | 24-38 | 153-209 | JOE (green) |
| D12S391 | 15-24 | 104-140 | NED (yellow) |
| THO1* | 4-13.3 | 56-95 | 6-FAM (blue) |
| FGA* | 17-51.2 | 124-262 | 6-FAM (blue) |
| TPOX* | 6-13 | 57-85 | JOE (green) |
| VWA* | 11-24 | 90-142 | JOE (green) |
| D5S818* | 7-16 | 119-155 | NED (yellow) |

*biotinylated primer.

Furthermore, to detect allele peaks of the other multiplex in case of poor biotin-streptavidin separation, the allele ladders do not overlap with respect to their tetramer repeat sizes between the 6-plex and the 5-plex reactions, when the same fluorescence dye is used.

Multiplex separation and capillary gel electrophoresis. The biotinylated products of the PCR reaction were immobilized on streptavidin-coated sepharose beads (Streptavidin Sepharose™ HP, Amersham Biosciences Ltd.). Briefly, three microlitres sepharose beads solution with 20 μl PCR products, binding buffer (10 mM Tris-HCl, pH 7.6; 2 M NaCI; 1 mM EDTA, pH 8.0; 0.1% Tween 20) and water in a final volume of 80 μl were mixed. The mixture was incubated for 15 minutes at room temperature under continuous mixing at a shaker (2000 rpm). After the immobilization the beads were centrifuged, the supernatant was further purified with the MSB Spin PCRapace Kit (Invitek GmbH, Germany) according to the instructions of the manufacturer, and the PCR products were finally collected in 20 μl elution buffer (10 mM Tris-HCl, pH 8.0). PCR products immobilized on sepharose beads were washed twice, first with 150 μl wash buffer (10 mM Tris-Acetate, pH 7.6) and then with the same volume of 70% ethanol. Finally, the beads were resuspended in 20 μl elution buffer from the MSB Spin PCRapace kit. Five microlitres of each purified PCR fraction were mixed with 25 μl formamide, containing 1.2 μl internal Spur Standard 600 (ILS600, Promega), and separated by capillary gel electrophoresis (by using POP-6 polymer) in an ABI PRISM 310 or 3100avant Genetic Analyzer (Applied Biosystems, Foster City, Calif., USA).

Specificity and reproducibility. To examine the specificity of the novel STR multiplex, 66 pg of non-degraded human DNA from 15 blood samples were at least twice amplified, separated and analyzed by capillary gel electrophoresis. The results were compared with the typing results using the SGM Plus™ (Applied Biosystems) or PowerPlex® 16 (Promega) kits and the single amplification of the D12S391 STR system [W. Waiyawuth, L. Zhang, C. Rittner, P. M. Schneider, Genetic analysis of the short tandem repeat system D12S391 in the German and three Asian populations, Forensic Sci Int 94 (1998) 25-31]. The inventors observed only 0.4% failure alleles and no extra allele among all analyzed samples. By typing of 100 pg of genomic DNA all alleles were shown correctly. After separation and purification of the two multiplexes no cross-contamination between the two smaller multiplexes was ever observed.

Sensitivity and degradation analysis. For validation of the test sensitivity samples down to 25 pg DNA, the equivalent of about four nuclei, were successfully typed compared to the standard amplification of 100 pg of DNA (FIG. 2). The use of DNA amounts higher than 100 pg often led to N and N+1 fragments, which can typically be found in case of over-amplification [R. Sparkes, C. Kimpton, S. Gilbard, P. Came, J. Andersen, N. Oldroyd, D. Thomas, A. Urquhart, P. Gill, The validation of a 7-locus multiplex STR test for use in forensic casework. (II), Artefacts, casework studies and success rates, Int J Legal Med 109 (1996)195-204]. Even with 25 pg human DNA only a few “failure alleles” were observed. With a lower DNA concentration the risk for failure alleles dramatically increased. The failures started to occur with 50 pg, and increased drastically at 12.5 pg. “Drop in” alleles were not observed. These failures reflect stochastic effects, when extremely low DNA amounts are used for the amplification.

To examine the amplification efficiency of highly degraded DNA, the inventors analyzed DNA which was previously prepared for their investigation of degraded DNA [P. M. Schneider, K. Bender, et al. STR analysis of artificially degraded DNA-results of a collaborative European exercise, Forensic Sci Int 139 (2004) 123-134, K. Bender, M. J. Farfan, P. M. Schneider, Preparation of degraded human DNA under controlled conditions, Forensic Sci Int 139 (2004) 135-140]. The artificially degraded DNA from the two human cell lines HepG2 and P118 was typed with the novel multiplex. A successful amplification using commercially available STR multiplex kits was only obtained for fragments of up to about 220 bp. No or only poor results were obtained for longer systems, like D2S1338 and FGA. In contrast, correct genotypes were obtained for all tested samples using the Bioplex-11.

Cases and heterozygote peak balance. To examine the novel multiplex test during more practical work, the inventors analyzed telogen hairs of real investigations. After multiplex STR typing loci with heterozygote genotypes were examined to assay the peak balance, failure alleles and other artefacts. The typing of 104 telogen hair samples from investigations led to 68 whole DNA profiles, 23 partial profiles and in 13 cases to no amplification results. From the successfully analyzed hair samples the number of failures and extra alleles was calculated from a total number of 1224 typed alleles with 4.8% and 8.7, respectively. The peak balance for each heterozygote STR system was calculated according to Whitaker and Gill [J. P. Whitaker, E. A. Cotton, P. Gill, A comparison of the characteristics of profiles produced mit the AMPF1STR SGM Plus multiplex system for both standard and low copy number (LCN) STR DNA analysis, Forensic Sci Int 123 (2001) 215-223] and is summarized in Table 3. The highest peak imbalance was found for D2S1338 and FGA, the STR systems with the largest amplification products, and was sometimes difficult to distinguish from Stotter peaks. The mean peak height ratio for all loci was 0.91.

TABLE 3
Heterozygote balances for all STR loci in hair samples (n = 104)

| Locus | Minimum | Mean | Maximum | SD |
|---|---|---|---|---|
| Amelogenin | 0.5 | 0.9 | 1.17 | 0.18 |
| D3S1358 | 0.36 | 0.78 | 0.96 | 0.36 |
| D2S1338 | 0.18 | 0.99 | 4.5 | 0.91 |
| D8S1179 | 0.69 | 1.18 | 2.1 | 0.63 |
| D21S11 | 1.04 | 1.14 | 1.24 | 0.14 |
| D12S391 | 0.13 | 0.49 | 0.69 | 0.31 |
| THO1* | 0.56 | 1.03 | 1.89 | 0.26 |
| FGA* | 0.24 | 1.08 | 3.65 | 0.95 |
| TPOX* | 0.25 | 0.75 | 0.97 | 0.16 |
| VWA* | 0.23 | 0.86 | 1.83 | 0.38 |
| D5S818* | 0.52 | 0.77 | 1.05 | 0.24 |

1. A method for typing an individual, comprising the steps of
a) providing genomic DNA of the individual,
b) amplification of at least two short tandem repeat (STR) loci of the genomic DNA using, if applicable labelled, pairs of amplification primers, wherein at least one primer is provided with a binding group,
c) separating the amplified STR fragments by means of the binding group in at least two fractions of amplificates, and
d) separate detection of the STR fragments of the fractions.

2. The method for typing an individual according to claim 1, wherein the individual is a mammal.

3. The method for typing an individual according to claim 1, wherein the STR loci are selected from the group consisting of D3S1358, D8S1179, D21S11, TH01, FGA, VWA, D2S1338, D12S391, TPOX, D5S818, D18S51, FES and amelogenin.

4. The method for typing an individual according to claim 1, wherein more than three STR loci are amplified.

5. The method for typing an individual according to claim 1, wherein the amplification is carried out using PCR and/or multiplex amplification.

6. The method for typing an individual according to claim 1, wherein the primers are labelled with a fluorescence dye.

7. The method for typing an individual according to claim 6, wherein the fluorescence dye is selected from the group consisting of 6-FAM, JOE, NED and PET (red).

8. The method for typing an individual according to claim 1, wherein the binding group is selected from the group consisting of biotin, streptavidin, a His tag, heat stable antigens and oligonucleotides.

9. The method for typing an individual according to claim 1, wherein the genomic DNA is derived from blood, blood constituents, sperm and/or telogen hairs.

10. The method for typing an individual according to claim 1, wherein the separation of the amplified STR fragments by means of the binding group comprises a solid phase which has biotin, streptavidin, antibody(antibodies) or complementary oligonucleotides immobilized thereto.

11. The method for typing an individual according to claim 10, wherein the solid phase comprises a membrane, sepharose beads or magnetic sepharose beads.

12. The method for typing an individual according to claim 1, wherein the detection of the STR fragments of the fractions comprises a length determination of the fragments.

13. The method for typing an individual according to claim 1, further comprising an identification of the individual on the basis of the detected STR fragments.

14. The method for typing an individual according to claim 1, wherein as primer pairs at least two pairs are used which are selected from the group consisting of SEQ ID NOs. 1 and 3; 4 and 5; 6 and 7; 8 and 9; 10 and 11; 12 and 13; 14 and 15; 16 and 17; 18 and 19; 20 and 21; and 22 and 23.

15. A diagnostic kit, comprising at least two pairs of STR primers for performing the method according to claim 1, optionally with further materials and excipients.

16. The method according to claim 1 used in the context of forensics.

17. The method for typing an individual according to claim 1, wherein the individual is a human.

18. The method for typing an individual according to claim 1, wherein more than five STR loci are amplified.

19. The method for typing an individual according to claim 1, wherein more than eight STR loci are amplified.

20. The method for typing an individual according to claim 1, wherein more than eleven STR loci are amplified.
